(19) United States
(12) Patent Application Publication
(10) Pub. No.: US 2025/0061988 A1
(43) Pub. Date: Feb. 20, 2025

(54) HEALTHCARE EXCHANGE SYSTEM AND METHOD

(71) Applicant: AllClear ID Health, Inc., Austin, TX (US)

(72) Inventors: James Richard Holland, IV, Horseshoe Bay, TX (US); Janine Moore, Austin, TX (US)

(73) Assignee: AllClear ID Health, Inc., Austin, TX (US)

(21) Appl. No.: 18/388,804

(22) Filed: Nov. 10, 2023

Related U.S. Application Data
(60) Provisional application No. 63/424,359, filed on Nov. 10, 2022.

(51) Int. Cl. G16H 10/60; G06F 9/54; G06Q 40/08; H04L 9/40

(52) U.S. Cl. CPC G16H 10/60; G06F 9/541; G06Q 40/08; H04L 63/0861; H04L 2463/082

(57) ABSTRACT

This disclosure relates broadly and generally to systems and methods for health data exchange platforms, and more particularly, the aggregation, formatting, management, storage, and accessing of a patient's health data. This includes, among other things, the management, access authorization, and electronic exchange of health information between patients and their providers. Health ID Network system and the methods of their use, as discussed herein, may provide for enhanced functionality, including but not limited to automation of the collection, formatting, consolidation, and storage of a patient's health records. They may also facilitate the transfer of sensitive personal health information between authorized parties and provide for patients to possess greater access to and control over their health records, which in turn may enable patients to become more actively involved in the management of their health information and provide for them to obtain better outcomes when interacting with providers, among other benefits.

### PRIORITY STATEMENT

### UNDER 35 U.S.C. § 119 & 37 C.F.R. § 1.78

This non-provisional application claims priority based upon prior U.S. Provisional Patent Application Ser. No. 63/424,359 filed Nov. 10, 2022, in the names of Bo Holland and Janine Moore entitled “HEALTHCARE EXCHANGE METHOD AND SYSTEM,” the disclosures of which are incorporated herein in their entirety by reference as if fully set forth herein.

### BACKGROUND OF THE INVENTION

The purpose of this disclosure is to provide the means to empower patients to take control of their medical records and act as the bridge between different providers, researchers, and payers. It's a portability model that has the power to transform healthcare.

Patients need the ability to share their records instantly wherever they need care, a second opinion, a prior authorization, or personalized medicine. When our doctors can't figure out what is wrong, they need their data to search for cures and participate in research.

A physician's knowledge of his or her patient is central to caregiving. The healthcare industry relies on the patient's most personal and detailed information to provide proper care and find cures. Healthcare providers dedicate tremendous time and effort to getting it right; a typical healthcare visit requires patients to provide identity and health information, followed by confirmatory questions from the healthcare professional. The process is intentionally redundant to match the patient to their existing profile in the electronic health record (EHR) system, and to ensure that the patient's records are accurate and up to date.

An electronic health record is a collection of electronically stored health information in a digital format. For example, EHR's may include a patient's demographics, medical history, medication and allergy information, immunization status, laboratory test results, medical imaging, insurance claims, personal statistics like age height and weight, billing information, and other personal identifiable an/or otherwise sensitive health information.

Historically, healthcare providers have zealously guarded their patients' health information not only due to confidentiality requirements such as HIPAA, but also because they effectively see such information as their proprietary trade secret assets. This has resulted the siloing of patient health care information into proprietary EHR systems, owned by different providers, which often do not prioritize intercommunication or patient accessibility, much less a patient's ownership and control of their own medical records and information. This has resulted in patients having highly fragmented medical records stored across multiple provider systems, each of which may be difficult to access, much less aggregate and obtain control over such that the patient can control access to, and the transfer of, their own medical records as needed between other providers. In these environments, the fragmentation and lack of portability of a patient's medical record results in multiple issues, including reducing the quality of care a patient may receive, while simultaneously increasing its cost.

Other issues in current medical records systems include those related to data processing, including the entry of incorrect information, misidentification of patients, use of non-standardized file formats, presence of inconsistent or conflicting information across records, etc. Data processing issues, such as those listed, may result in the loss of a patient's medical information, entry of inaccurate medical information into a patient's record, denial of claims, and loss of payment for medical services rendered.

The root causes of missing clinical information and patient misidentification include, but are not limited to:
record fragmentation across different EHR systems;
inaccurate or incomplete data provided by the patient;
weak identity proofing and authentication;
patient record mismatching and duplication; and
data entry errors.

Mistyped, outdated, or incorrect information tied to an instance of care gives payors grounds to deny the accompanying claim. In as many as 93% of cases, the provider reworks and resubmits the claim successfully, costing approximately $25 on average. In the remaining cases, the provider is left on the hook for procedures that were redundant or unnecessary due to information that did not make it into their EHR system. It costs the average hospital $29 million to rework or forfeit claims for every 1 million patients it treats annually. Fully one third of all initially denied claims can be sourced to mistyped biographical information, outdated information, erroneous matches to the wrong record, or matches to duplicate records that tell incomplete stories.

While health records themselves are now mostly electronic, the dominant patient interface is still paper forms and plastic insurance and ID cards. Providers operate EHR systems with analog identity credentials. In most care settings, a patient is matched to their records in an EHR system by filling out paper forms, showing plastic ID and insurance cards, and answering biographical questions either verbally or via more paper forms. At check-out, providers deliver information to patients via documents, phone calls, CDs, and thumb drives. This approach drives the two biggest patient complaints: repetitive form filling and difficulty gaining access to their medical records. Additionally, on the provider's side, most patient record lookups and data exchanges are manual which is expensive, frustrating and error prone.

Problems are compounded when a health system operates more than one HER system. Often separate facilities within the same health system run disjointed EHR systems, and when a patient's journey crosses EHR systems, they must fill out the same forms and registrars must complete the check-in steps multiple times. This redundancy and duplication of effort not only results in inefficiency, but means a patient has a different medical record number (MRN) stored in each care setting, and critically, healthcare providers may have no view of their patient's records in EHR systems other than their own.

For providers, this environment limits their view of their patients' healthcare record to the narrow cross section captured by that provider's specific EHR system. Information in each EHR system, including that of the provider, may not always be complete, up to date, or accurate which can result in suboptimal patient service, including but not limited to, misdiagnoses, delayed diagnosis, receipt of incorrect treatments, denial of insurance claims, etc.

Moreover, this model burdens the patient to correctly identify themself, demonstrate their insurance eligibility, and repeatedly summarize their medical history at each point of care process. The patient must also act as a middleman in keeping the EHR system of each provider with which she interacts consistently up to date, which can in some cases constitute the physical carrying of thumb drives, binders of physical records, and/or CDs between care facilities. This effectively results in patients having relatively minimal access to their medical records, while at the same time, the responsibility for maintaining the accuracy of their records is being forced upon them.

Additionally, as different providers use different proprietary system to store their patients' health information, it is not uncommon for even digital health records to be stored in various formats, many of which may not be compatible with software used by other providers, or which may not be consistent with best practices or regulatory standards. The need to manually translate data between formats presents yet another time at which inaccuracies may be introduced to the record, by means such as simple human error (e.g., clerical mistakes, etc.).

Regulatory standards, including specific formatting requirements, have been developed to help facilitate the exchange of health information between authorized parties including patients, providers, and payors; however, many historic records still need to be converted to such formatting in order to adhere to such regulatory standards and best practices. Accordingly, systems that are capable of accepting health records across a multitude of formats, and which can automatically and accurately convert such records into standard formats that correspond to regulatory standards and/or best practices would be beneficial. This is especially true for systems that both aggregate and are responsible for further disseminating such covered information between authorized parties.

With medical records being created at different times by different parties, it is not uncommon for an individual's medical records to have inconsistencies or instances of outright incorrect information included therein. Such inconsistent and/or inaccurate information in a patient's record also may result in the patient obtaining less than optimal service from providers.

Use of systems that provide for the normalization of information across a patient's medical records received from different providers may help alleviate the burden on the patient to ensure the consistency of information across their medical records. A system that can automatically identify and correct inconsistencies and errors in a patient's healthcare records would greatly assist in relieving this burden from patients. Additionally, the reformatting of medical records into a standardized formatting may enable such identification and correction to be performed much more easily than if it were to be tried across records having inconsistent formatting standards.

Many current healthcare information systems connect patients to healthcare information with low security, static identities that can fail both providers and the patient. This is often the weakest link in security during the patient experience. Government issued IDs and patient portals do not necessarily completely secure the patient's data. Government IDs are mobile, meaning they can grant healthcare access at any facility that the patient needs, but fail to carry any context about the patient's health or demographic information, opening the door for patient misidentification and repetitive paperwork. Patient portals address those problems but have historically been cripplingly immobile and reactive.

Web/application-based portals may address many of these problems and represent a big step forward in the healthcare industry generally but are only a piece of the puzzle. In cases where providers have adopted electronic identity credentials, e.g., patient portals, to solve their security concerns, the misidentification problem may be only partially addressed while new cybersecurity risks may be introduced. Such application-centric identity systems are typically protected by username and password, sometimes with one-time passcodes, which may leave patient data and the EHR system itself vulnerable to attack.

It is well known that username and password designs are relatively easy to defeat and provide no actual assurance that the party using those credentials to access a portal is the person authorized to access the portal using those credentials (i.e., it could be an unauthorized party who was given, or stole, the credentials). Additionally, such credentialing and authentication systems contain stagnant information that the patient is made to be responsible for maintaining, potentially allowing mistyped and outdated information to enter the patient's record. Moreover, while these systems may be capable of making a 1:1 match between a patient and their MRN, they do so only in the silo of the EHR system for which they were built.

In addition to the challenges described above, the healthcare industry is increasingly under siege by ransomware and fraud attacks. Application-centric patient identity systems are weakly protected gateways by which such criminals may access EHRs and gain patient information. Medical service providers store peoples' most personal, sensitive, and important information. The theft of, or restriction of a healthcare provider's access to, a patient's medical records by a third party may result in serious issues, including but not limited to invasion of a patient's right to privacy and theft of the patient's identity. Further, when a hospital's systems are crippled by ransomware attack, patient safety is jeopardized. Criminals recognize and exploit the value of the information in healthcare systems, and in keeping those systems up and running, and therefore are willing to threaten them in order to obtain leverage over another party. The National Institute of Standards and Technology recently deprecated one-time passcodes for secure authentication because of their susceptibility to man-in-the-middle attacks, and low levels of authentication for patients and providers also leave a door open to exploitation by other means, such as by way of social engineering attacks.

The only solution that can meet these requirements today are high security multifactor authentication protocols, such as those that are regularly used with mobile devices. Authentication Assurance Level 2 (AAL2) is the recommended standard, which can be achieved and surpassed with mobile multi-factor authentication. High security multifactor authentication protocols also provide a secure means to exchange data electronically with EHR and health information exchange systems. Accordingly, such multifactor authentication protocols may be safe to reuse across different EHR systems as well as in other healthcare-related settings and may travel with the patient who is the ultimate source of truth and steward of their information.

As mentioned previously, medical records and certain other personal information regarding patients, are governed by specific regulations, including HIPPA, which require that such information be stored and transmitted in accordance with established requirements to ensure at least a minimum level of privacy for such information. Accordingly, any systems dealing in the storage and exchange of information covered by such regulations, including those contemplated in this disclosure, must meet or exceed such requirements in order to be compliant with HIPPA.

The aforementioned siloing of patient health information across a multitude of disparate proprietary EHR systems and the simple fact that health record information storage systems have changed through the years, from paper records to different image and digital document storage formats, has made it difficult to consolidate and transfer these records between patients and providers in a consistent manner that allows for easy and secure transfer of such records in a manner compliant with current standards. Recently, additional pressures have been placed on EHR systems to innovate to comply with the 21st Century Cures Act, which challenges providers to meet new standards to facilitate the exchange of health information. This requires the interoperability of EHR systems, which is a key enabler for a future healthcare information exchange systems to interact with a wide range of EHRs such as through standardized APIs.

The COVID-19 pandemic has accelerated the movement towards digitized healthcare. There has been significant growth in electronic healthcare services and patient use of telemedicine and other healthcare technologies, including mobile apps, indicating strong incentives for provider innovation in this space. The pandemic exposed a need for there to be the capability of real-time, accurate, healthcare screening at the individual level. During the pandemic, the U.S. government issued individuals paper vaccination cards on which they were to have provider signoff after receiving vaccines for the COVID-19 virus. Individuals where then required to present these documents to officials when traveling or attending events. Obviously paper documentation, with minimal security features or other means of having either the document itself or the signatures indicating the receipt of vaccinations thereon, carried in the pocket of an individual has several potential issues. Such documentation can easily be lost, destroyed, or forged. The use of a health information system that provides patients with ownership of their own health information, including the ability to immediately access authenticated medical records, may serve to solve issues like those related to real-time in-person health screening, such as was instituted responsive to the COVID-19 pandemic, and may enable such actions to not only be performed both more easily and with a much higher level of certainty.

Outside of the healthcare space, people have grown accustomed to mobile automation in daily life to access contact-free services from their bank, restaurants, and other businesses through web portals, mobile apps, QR codes, etc. Similar 21st century technologies may be leveraged in order to provide individuals with constant, secured, access to their own validated medical records and health information, which may be accessed in real-time from any of multiple user devices using high security multifactor authentication protocols. This would provide patients with greater ownership of their medical information and would provide their medical service providers with more accurate, complete, up-to-date, and trustworthy information regarding the patient, resulting in the improvement of services rendered to the patient by the provider and a corresponding reduction in any risks which may result from providing the patient with inferior healthcare services.

Accordingly there is a need for a secure healthcare information exchange system that:
collects medical records from a multitude of providers;
converts the healthcare information so received into standardized formats;
identifies and corrects inconsistencies in patient information across the patient's health records;
protects patient information using high security multifactor authentication protocols;
stores and transfers information in a manner that complies with relevant regulations and standards; and
provides patients with ownership of their authenticated health information.

### SUMMARY OF THE INVENTION

The present invention relates broadly and generally to systems and methods for health data exchange platforms, and more particularly, the aggregation, formatting, management, storage, and accessing of a patient's health data. This includes, among other things, the management, access authorization, and electronic exchange of health information between patients and their providers.

Certain arrangements of structural aspects of the present invention, forming systems for the storage and exchange of EHR, are described in detail below, and may be referred to herein by various means such as the “Health ID Network”, the “Health ID Network system,” the EHR system, and/or the system. Certain presently preferred embodiments of the Health ID Network system and of certain method of its use are discussed in detail below. It should be appreciated, however, that the present invention provides many applicable inventive concepts that can be embodied in a wide variety of contexts other than the specific types of exchanges described herein. Accordingly, the specific embodiments discussed are merely illustrative of specific ways to make and use the invention, and do not limit the scope of the invention.

Embodiments of a Health ID Network system may include a server, a database, and one or more software applications through which a party may transmit information to, and receive information from, the server using a sure device. The components of the system may be connected via a suitable network and may or may not be geographically distributed.

Such systems may include software, which when executed causes them to perform certain functions required to aggregate, consolidate, process, store, and manage access to a patient's medical information. Such functions including, but not limited to, to obtaining patient authorization/consent to transfer medical information; requesting medical records from providers on behalf of the patient (using the patients' authorization/consent when necessary); associating records received form a provider with the patient's account; processing the records to convert them into a standardized format; processing the records to identify and cure discrepancies, inconsistencies, or errors in the health records; and transmitting the records to the patient and/or authorized third parties, which may be performed via one or more of software application(s) running on a user device, portal(s) accessible from a remote device via a network, and software API(s) connecting the system to a provider software platform.

According to HIPPA requirements, to access/obtain data on behalf of a patient, the Health ID Network should be authorized by that patient via a medical records release form addressed to the provider that owns the records. To eliminate additional dependency and frustration from the patient, embodiment Health ID Networks may be configured with established workflows to automatically address known requirements for the transfer of information known to be covered by applicable standards/regulations. Such automated workflows may include, for example, instructions to request a blanket authorization from the patient to create documents designed to meet such requirements on their behalf. The system may transmit such a request from the server to the patient application, and the patient may provide their approval of such an authorization in a manner whereby the identity of the party providing the authorization may be individually authenticated via said patient application. The Health ID Network system may then forward an authorization/consent along with request(s) for the patient's medical records to one or more members of their care team including providers, payors, researchers, advocacy organizations, the patient's friends or family members, etc.

Embodiments of Health ID Network system may be configured with software which when executed causes the system to request and collect other patient-related information (e.g., insurance claims history, financial records, mobile device location data, etc.) from a provider, and to use the other patient-related information so captured to identify additional providers and/or events which may be linked to additional providers, which may have additional medical records on the relevant patient. The system may then be configured to automatically send records requests to the additional providers so identified to capture the patient's health records and information from those providers, integrate the patient's captured information with the system's existing records for the patient, and thereby develop a more complete and comprehensive medical record for the patient, which the patient or another duly authorized party may then access and/or manage via an authenticated session inside of a software application using a mobile device.

Embodiments of Health ID Network system may be configured with software which when executed causes the system to automatically convert patient health records received from a provider to a standard format, which may be compliant with applicable regulations/standards.

Embodiments of such systems may also be configured such that they automatically normalize patient health records received from provider against the system's other health records for the patient. Normalization may include the identification of inconsistencies in information across medical records, and in some implementations may include the automated correction of a patient's medical information and/or records based on the patient's other medical records in the system.

The use of the systems and methods discussed herein; namely the Health ID Network and methods of its use, may provide solutions to many of the problems hindering the current health information exchange paradigm. Such systems and methods may (i) provide for the aggregation and consolidation of a patient's health records from across various disparate provider silos to provide a more comprehensive medical record for the patient; (ii) normalize the information in that record to identify and cure defects therein which may lead to inaccurate diagnosis, improper treatments, or other suboptimal outcomes for the patient; (iii) consolidate and standardize the records and the information contained therein into formats that are compliant with relevant regulations and the industries' best practices; and (iv) facilitate the easy and secure exchange of such information between authorized parties, including the patient, thereby providing the patient with ownership of their own medical information. Such patient ownership facilitated by the system may include its ability to provide the patient with immediate access to their own authenticated health records, and active control over the portability of those records.

Additionally, such systems may facilitate the automation of such collection and transfer of patient health information by leveraging automated workflows to meet known requirements for the processing of such health records and information, such as by automatically generating the consents/authorizations and records requests required to collect and/or transfer patient health records.

Further, Health ID Network system may be configured to use high-security multifactor authentication protocols to prevent unauthorized access to the system and the information contained therein, and to ensure the authenticity of any requests, authorizations, and/or consents issued through either the software application operating as the patient's point of access to the system, or an access portal which may provide similar functionality for providers. In embodiments, such multifactor authentication protocols used by the system may include biometrics. Such embodiments may allow for parties to be authenticated at an individual, personal, level using their biometrics. Such individualized authentication may provide the system with additional functionality, including but not limited to the ability to authenticate the individual identity of the patient when they are performing certain functions for which such authentication of the patient's identity may be required, such as when providing authorizations/consents for the transfer of their medical records.

The foregoing has broadly outlined certain aspects of the present invention in order that the detailed description of the invention that follows may better be understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### DESCRIPTION OF THE DRAWINGS

For a more complete understanding of the present invention, and the advantages thereof, reference is now made to the following descriptions taken in conjunction with the accompanying drawings, in which:

FIG. 1 provides a diagram of an exemplary Health ID Network EHR system, in accordance with embodiments;

FIG. 2 provides a diagram of an alternate embodiment of an exemplary Health ID Network system, in accordance with embodiments;

FIG. 3 provides an alternate system diagram of an exemplary Health ID Network system, in accordance with embodiments;

FIG. 4 provides a flowchart depicting an exemplary method of providing a requesting party with access to patient records using a Health ID Network system, in accordance with embodiments;

FIG. 5 provides a flowchart depicting an exemplary method of providing a requesting party with access to patient records using a Health ID Network system, as seen from the perspective of the system's server, in accordance with embodiments;

FIG. 6 provides a flowchart depicting an alternate exemplary method of providing a requesting party with access to patient records using a Health ID Network system, in accordance with embodiments;

FIG. 7 provides a flowchart depicting an alternate exemplary method of providing a requesting party with access to patient records using a Health ID Network system, as performed from the perspective of a Health ID Network system server, in accordance with embodiments;

FIG. 8 provides a flowchart depicting an exemplary method for automatically collecting patient health records based on other patient-related information (e.g., insurance claims history, financial records, mobile device location data, etc.) using a Health ID Network system, in accordance with embodiments;

FIG. 9 provides a flowchart depicting an alternate exemplary method for automatically collecting patient health records, as performed from the perspective of a Health ID Network system, in accordance with embodiments;

FIG. 10 provides a flowchart depicting an exemplary method for automatically normalizing and consolidating health records received by a Health ID Network system, in accordance with embodiments;

FIG. 11 provides a flowchart depicting an alternate exemplary method for automatically normalizing and consolidating health records received by a Health ID Network system, as performed from the perspective of a Health ID Network system server, in accordance with embodiments;

FIG. 12 provides a flowchart depicting a method for implementing an exemplary automated workflow for automatically authorizing requests for the transfer of a patient's medical records to a Health ID Network system, in accordance with embodiments; and

FIG. 13 provides a flowchart depicting an alternate exemplary method for implementing an exemplary automated workflow for automatically authorizing requests for the transfer of a patient's medical records to a Health ID Network system, as performed from the perspective of a Health ID Network system server, in accordance with embodiments.

### DETAILED DESCRIPTION

The present invention is directed to improved methods and systems for, among other things, the secure storage and exchange of sensitive medical data between authorized parties across a network. The configuration and use of the presently preferred embodiments are discussed in detail below. It should be appreciated, however, that the present invention provides many applicable inventive concepts that can be embodied in a wide variety of contexts other than networks for the storage and exchange of medical information. Accordingly, the specific embodiments discussed are merely illustrative of specific ways to make and use the invention, and do not limit the scope of the invention. In addition, the following terms shall have the associated meaning when used herein:
“application” is a software program designed to run on or be accessed and operated by a user device;
“cloud” means a collection of logical devices which may or may not include underlying physical servers, wherein all such logical devices may be accessed without any knowledge, or with limited knowledge, of the underlying physical devices, and wherein the collection of logical devices has persistent logical resources, but is non-deterministic in its use of physical resources;
“payors” means a party that pays for healthcare services provided to a patient (e.g., insurance providers);
“portal” is a software program hosted on a device other than a user device, through which a party may access a hardware and/or software system via a remote user device;
“provider” means a healthcare service provider, who may possess patient health records and/or other materials containing patient health information (e.g., doctors, hospitals and their staff, etc.), and includes payors;
“storage” means a non-transitory electronic storage device, such as an electronic database, which may be network-based; and
“user device” means any computing device comprising a graphical user interface and a user input of which a patient or provider has local access and/or control, including but not limited to desktop computers, laptop computers, tablets, smartphones, etc.

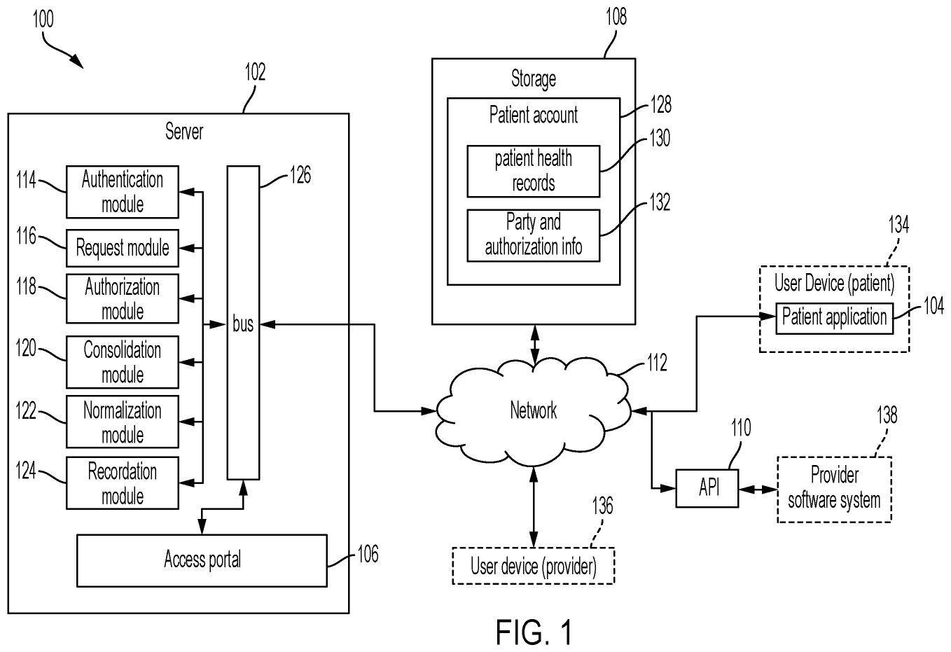

FIG. 1, provides a diagram of an exemplary Health ID Network EHR system, namely system 100. Health ID Network systems, such as system 100 may facilitate the secured transfer of sensitive medical information, including health records covered by HIPPA, between multiple authorized parties.

System 100 may comprise server 102, patient application 104, access portal 106, and storage 108. System 100 may rely on a suitable communications network (e.g., via the internet and/or a private network), such as network 112, to enable the transmission of requests and information between its constituent components, as well as with user devices.

Server 102 may be configured to request patient health records from providers on behalf of the patient, and to record health information so obtained on, and to retrieve such health information from storage 108.

In embodiments, server 102 may comprise a plurality of operating modules, including but not limited to authentication module 114, consolidation module 120, normalization module 122, authorization module 118, and request module 116, each of which may be configured to communicate with each other and with the other constituent components of system 100 via one or more of bus 126 and/or network 112.

In embodiments, a party's (the patient, a provider, a payor, etc.) ability to access system 100 and/or information recorded in storage 108, including patient health records 130 and party and authorization information 132, via one or more of patient application 104, access portal 106, and/or API 110, may require that party's identity and authorization to be validated using a multifactor authentication protocol.

Information stored in patient account 128, (e.g., party and authorization information 132), may include one or more multifactor authentication credentials associated with parties associated with the patient account.

In embodiments, authentication module 114 may be configured to receive and validate the identities of parties accessing system 100 responsive to receipt of one or more multifactor authentication credentials submitted to system 100 by a party via a user device, through one or more of patient application 104, access portal 106, and API 110.

In such embodiments, a party may submit multifactor authentication credentials to system 100 from a user device (e.g., patient user device 134, provider user device 136, or provider software system 138) via patient application 104, access portal 106, or API 110. Authentication module 114 may receive the multifactor authentication 128 credentials so supplied, compare them against information stored in system 100, including party and credentialing information 132, which may be recorded in storage 108 and associated with patient account 128, and validate an identity of the party submitting the credentials.

Depending on the access authority of a given party, which may itself be associated with patient account 128 and recorded on storage 108, system 100 may provide for a patient's health information 130 to be accessed or otherwise shared by server 102 through one or more of patient application 104 and access portal 106.

In embodiments, the multifactor authentication credentials associated with the patient may comprise at least one biometric authentication factor, wherein the biometric authentication factor is based on the patient's own biometric information. By requiring at least one factor of at least the multifactor authentication credentials necessary for patient access to system 100 to be biometric and to specifically match the biometrics of the patient associated with the patient account, system 100 may verify the patient's actual identity. By verifying the individual identity of the party accessing system 100 via patient application 104 as the patient using biometric authentication factors comprising the patient's biometrics, system 100 may provide for the creation and exchange of verifiably valid and enforceable releases and consents, as maybe required by relevant standards/regulations governing patient health information storage and processing.

In addition to being able to verify a party's identity, system 100 must be able to ascertain whether a party making a request, such as a request to access or transfer patient health records 130 recorded in storage 108, possesses the requisite authorization to have the request fulfilled. In embodiments, authorization module 118 may receive party and authorization information 132 associated with multifactor authentication credentials received by system 100 via one of patient application 104, access portal 106, or API 110, to determine the authorizations that system 100 already has stored, such as party and authorization information 132, for the party identified via the submitted authentication credentials.

In embodiments, authorization module 118 may be configured to compare the requesting party's stored authorizations against the authorization required to complete the request submitted by that party. If authorization module 118 determines that the party making the request's authorization meet or exceed those required to perform the requested action, authorization module 118 may permit the request. If authorization module 118 determines that the requesting party's authorizations fail to meet those required to perform the requested action, authorization module may terminate the request.

A primary role of system 100 is to collect patient health information from providers and other third-party sources. To do this, system 100 must be configured to generate and transmit suitable records requests, including ancillary documentation like patient and/or provider consents/approvals, when necessary.

In embodiments, request module 116 may be configured to automatically generate records requests and ancillary documentation and to transmit those requests to the patient, providers, payors, and/or other third-parties by suitable means, including but not limited to via patient application 104, access portal 106, API 110, email, facsimile, or other means known in the art.

Embodiment of request module 116 may also be configured to receive records requests from one or more of patient application 104, access portal 106, and API 110, and if the request is approved by authorization module 118, request module 116 may fulfil the request, such as by providing the requesting party access to the patient's health records 130 on storage 108. Additionally, request module 116 may be configured to pass records request received from one party (e.g., the patient via patient application 104) to another party (e.g., a provider via access portal 106).

System 100 may be configured to receive and store patient heath information responsive to records request generated or forwarded to a party by request module 116. Once system 100 receives patient health records, those records may be recorded in storage 108 and associated with patient account 128 via recordation module 124; however, when initially received, the patient records may not be in preferred, standardized, and/or consolidated formats. If that is the case, in embodiments, consolidation module 120 may be configured to receive the patient records, convert them from one or more original formats to one or more standardized and/or consolidated format(s), and then may record the records in the standardized/consolidated format(s) on storage 108 in association with patient account 128 via recordation module 124. In embodiments, such standardized/consolidated format(s) may include but are not limited to those consistent with C-CDA, DICOM, and FHIR standards, and those which are compliant with HIPPA regulations.

In addition to converting the patient's records to a standardized/consolidated format and storing them that way, embodiments of the system may benefit from normalizing the information contained in any recently received records against the rest of the patient's records available to the system. To achieve this, embodiments of the system may provide for normalization module 122, which may be configured to compare patient information contained in records recently received by system 100 against the rest of the patient's records 130 in system 100, and to identify inconsistencies between the newly received patient records and those already in the system.

In embodiments, normalization module 122 may be further configured to automatically replace a portion of the information contained in a recently received patient record responsive to both the determination of an inconsistency between the information in the recently received records and those in the system's other records for that patient, and a determination that such a correction of the inconsistency is appropriate. The corrected records may then be saved to storage 108 and associated with patient account 128 via recordation module 124.

In embodiments, the determination of the appropriateness of correcting an inconsistency in the recently received patient record identified by normalization module 122 may be based one or more rule sets, each of which may be customized for any individual data element and/or patient. For example, if system 100 receives a patient's medical records from a first provider indicating that the patient has a shellfish allergy, and receives medical records from a second provider showing the patient has an allergy to shrimp, normalization module 122 may, after identifying the inconsistency between the records, apply rule sets related to, for example allergy identification and classification, to determine how such conflicting diagnoses should appear in the patient's consolidated record (e.g., patient health records 130). Normalization module 122 may then modify one more of the records accordingly. Further embodiments may provide for such rule sets to be created and/or modified by a provider having appropriate authority.

Embodiments of recordation module 124 may be configured to receive information comprising patient health records and information, as well as account, party, and authorization information from the other constituent components of system 100, and to record such information in storage 108.

In embodiments storage 108 may be configured to be compliant with the information storage and transfer requirements of one or more regulatory frameworks and/or data format standards, including but not limited to HIPAA, GDPR, DICOM, FHIR, and C-CDA.

In embodiments, such as that of system 100, access portal 106 may be hosted on server 102 and may be configured to be accessed by a user device, such as provider user device 136, via network 112. Access portal 106 may be used by healthcare service providers such as doctors, hospitals, insurance providers, and payors, to send health information and associated requests to, and to receive to receive health information and associated requests from, server 102.

In embodiments, such as that of system 100, patient application 104 may be loaded onto and executed by a user device associated with the patient, such as patient user device 134, and may communicate with server 102 via network 112. Execution of patient application 104 on patient user device 134 may cause patient application 104 to send requests to, and receive requests from, server 102.

Some embodiments of Health ID Network systems may additionally comprise one or more API(s), such as API 110, which may be configured to facilitate the secure transmission of sensitive health information between server 102 and a provider/payor software system, such as provider software system 138.

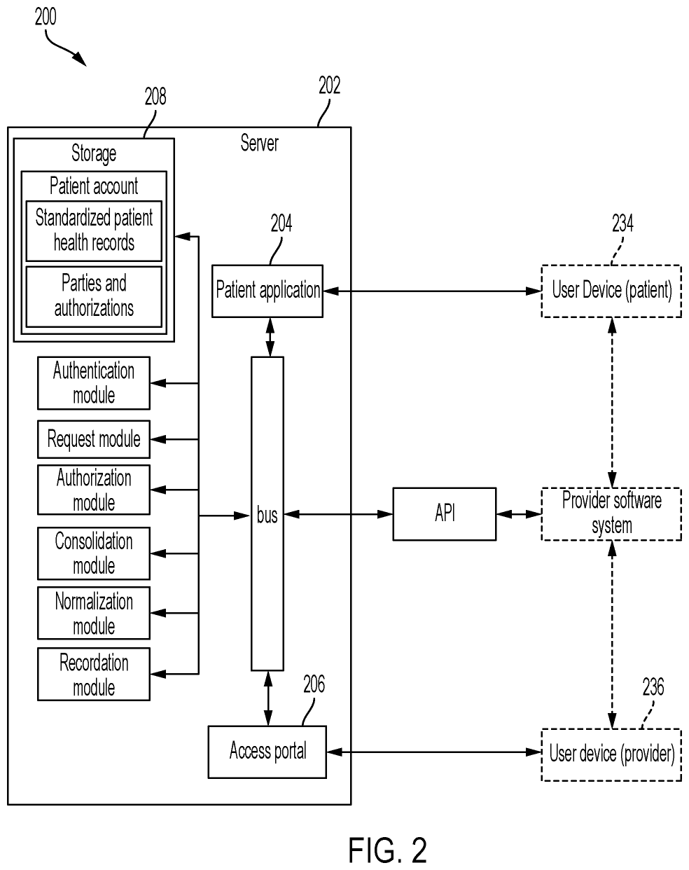

FIG. 2 provides a diagram of an alternate embodiment of an exemplary Health ID Network system, namely system 200, wherein the patient application and storage components of the system have been consolidated into the server rather than being hosted remotely therefrom. In the embodiment of the system so depicted, patient application 204 may be installed on and executed by server 202, as opposed to being stored and executed locally on patient user device 234 (as is seen in system 100 of FIG. 1). In such embodiments, patient application 204 may be configured to be remotely accessible by, receive information and requests from, and send information and requests to, patient user device 234 via a network. Essentially, in such embodiments, patient application 204 operates for patient user device 234 in much the same way as access portal 206 operates for provider user device 236.

Similarly, in the embodiment of an exemplary Health ID Network system shown in FIG. 2, storage 208 may be integrated into server 208 rather than being hosted remotely from server 202 and requiring a network connection to pass information therebetween (as is also seen in system 100 of FIG. 1).

Notwithstanding the foregoing structural differences between the embodiments of exemplary Health ID Network systems depicted in FIG. 1 and FIG. 2 (e.g., the hardware storing and executing the software comprising the patient application and the relationship between the systems' servers and storages), the operation of such systems are essentially consistent with one another.

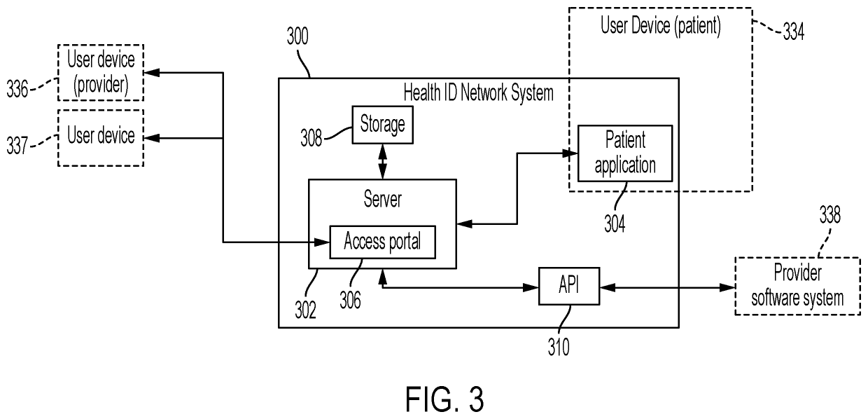

FIG. 3 provides a diagram of an alternate system diagram of an exemplary Health ID Network system, namely system 300, wherein system 300 comprises server 302, patient application 304, access portal 306, storage 308, and API 310. Patient application 304 may operate on patient user device 334 and may provide for a patient to access system 300 using said patient user device 334. Access portal 306 may operate on server 302 and may allow for provider(s) to access system 300 via user mobile devices (e.g., user device 336 and user device 337). Similarly, API 310 may enable provider access to system 300 via provider software system 338, which may be connected thereto.

In embodiments, such as the one depicted in FIG. 3, API 310 may be hosted remotely from the other component elements of system 300. Alternate embodiments may provide for API 310 to be hosted on server 302.

It may be noted that the system diagram of FIG. 3 and that of FIG. 1 depict a consistent embodiment of a Health ID Network system, just with the relationship between components of the system and the devices that interact with the system consolidated in a different manner.

According to HIPAA requirements, a patient should have the ability to see who has access to their medical records. Accordingly, embodiments of Health ID Network systems may be configured to receive a request for identification of parties with authorization to view each of a patient's medical records stored in the system, responsive to a patient request submitted via the patient software application. Health ID Network systems may be further configured to revoke, reject, or otherwise invalidate, any such parties' authorization to access one or more of the patient's medical records responsive to a patient's request to do so, which may be received via the patient software application.

Embodiments of Health ID Network systems may allow for patients to specify access permissions for portions of their health data stored in the system. Search permissions may be limited to certain documents or documents related to certain events or medical conditions or may otherwise comprise access limitations. In embodiments Health ID Network systems may allow for a patient to modify the status of their access permissions if, and only if, the system has validated multifactor authentication credentials received from the patient application submitting the change request.

Now that several exemplary embodiments of Health ID Network systems have been described, we may now turn to discussing methods in which such systems may be used and/or operate to provide particular functionality.

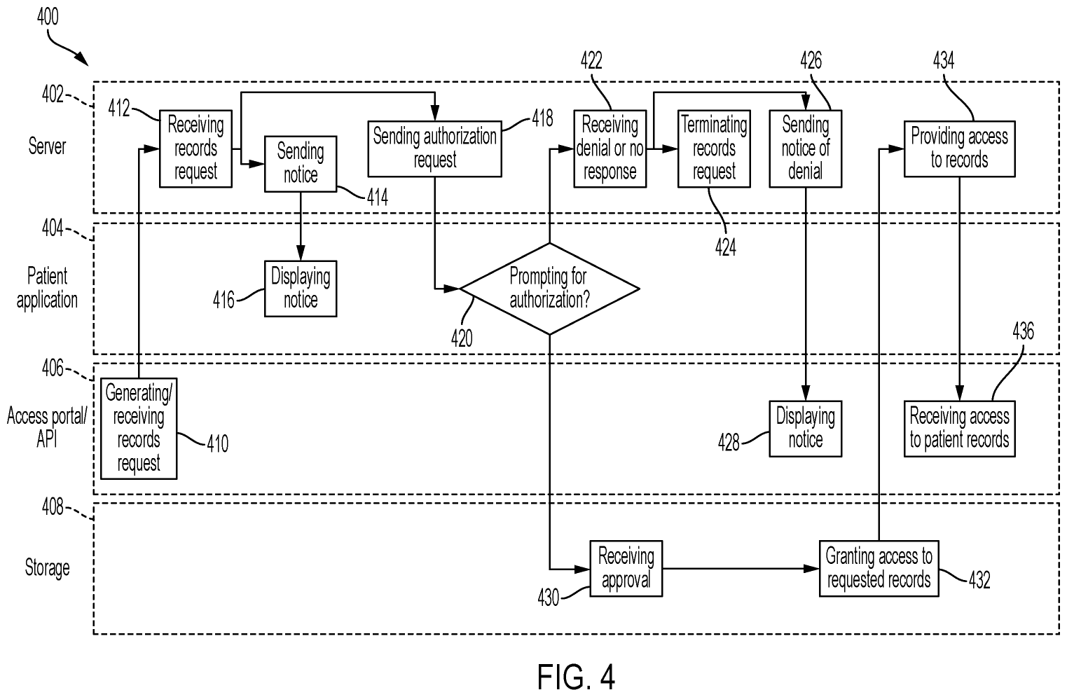

FIG. 4 provides a flowchart depicting an exemplary method of providing a requesting party with access to patient records using a Health ID Network system, namely method 400.

Method 400 comprises generating/receiving 410 a records request at access portal/API 406; receiving 412 the records request from access portal/API 406 at server 402; sending 414 a notification of the records request to patient application 404 and displaying 416 the notification on a patient user device via patient application 404; sending 418 an authorization request consistent with the received records request to patient application 404; and prompting 420 a patient for approval of the authorization request via patient application 404. To ensure that the party from which the system is receiving authorization is a party with the authority to validly provide such authorization, in embodiments, prompting step 420 may comprise the validation of the prompted party's multifactor authentication credentials to confirm the party's identity and review of the system's stored authorization information associated with that party.

Responsive to receiving 422 either a denial of the authorization request or a lack of response to prompting 420 (i.e., not receiving an approval of the authorization request), server 402 may perform the actions of terminating 424 the records request, sending 426 a notice regarding failure to receive approval of the authorization request to access portal/API 406, and displaying 428 the notification on a provider user device via access portal/API 406. Alternatively, responsive to receiving 430 an approval of the authorization request responsive to prompting 420, the system may perform the actions of granting 432 access to patient records covered by the records request in storage 408, providing 434 access to the covered records via server 402 and access portal/API 406 to the party that performed the step of generating/receiving 410 a records request via access portal/API 406, and the requestor may thereby receive 436 access to the requested patient records via access portal/API 406.

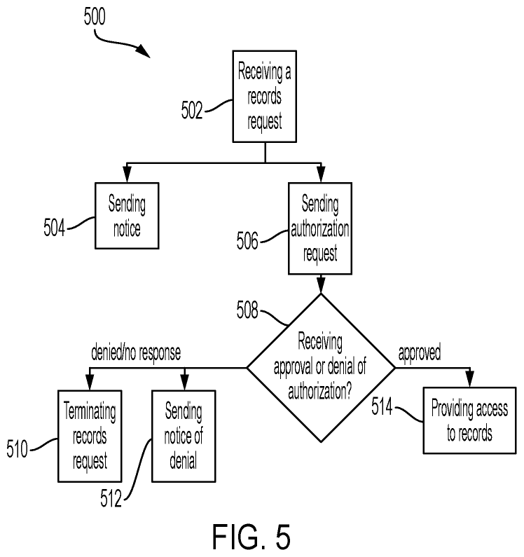

FIG. 5 provides a flowchart depicting an exemplary method of providing a requesting party with access to patient records using a Health ID Network system, as seen from the perspective of the system's server, namely method 500.

Method 500 comprises; receiving 502 a records request, which may be communicated to the server via one or more of a patient application, an access portal, or an API; sending 504 a notification of the request to a patient application; sending 506 an authorization request consistent with the received records request to the patient application; receiving 508 at least one of an approval of the authorization request and a lack of approval of the authorization request. Responsive to the lack of receipt of an approval of the authorization request at step 508, the system may take the actions of terminating 510 the records request and sending 512 notice of the denial to an access portal. Alternatively, responsive to the receipt of an approval of the authorization request at step 508, the system may perform the step of providing 518 the requesting party with access to the requested records via the access portal.

It should be understood that the methods of authorizing access to a patient's medical information stored in a Health ID Network system discussed above may also be operable for authorizing the transfer of such information between parties.

Further, the methods described above have been discussed in the context of authorization for access to or transfer of medical records being provided by a patient through the patient application; however, Health ID Network systems may provide for other methods, perfumed in a similar manner, but wherein instead of just the patient sending and receiving requests via the patient software application, another authorized party (e.g., a HCP, hospital administrator, or other provider, etc.) may receive and send the relevant requests through their access portal. In such embodiments, a records request, such as the one in the method to obtain a patient's medical records from a provider, as described above, may be submitted to a provider having the authorization to authorize such a transfer of said records via an access portal instead of being submitted to the patient via the patient software application.

In embodiments, a provider with suitable authorization over a patient's medical information may authorize a third party (e.g., another provider or a payor) with access to portions of the patient's medical records stored in the system. For example, a primary physician may authorize a consulting physician to access the patient's x-ray imaging for the purposes of consultation, receiving a second opinion, etc.

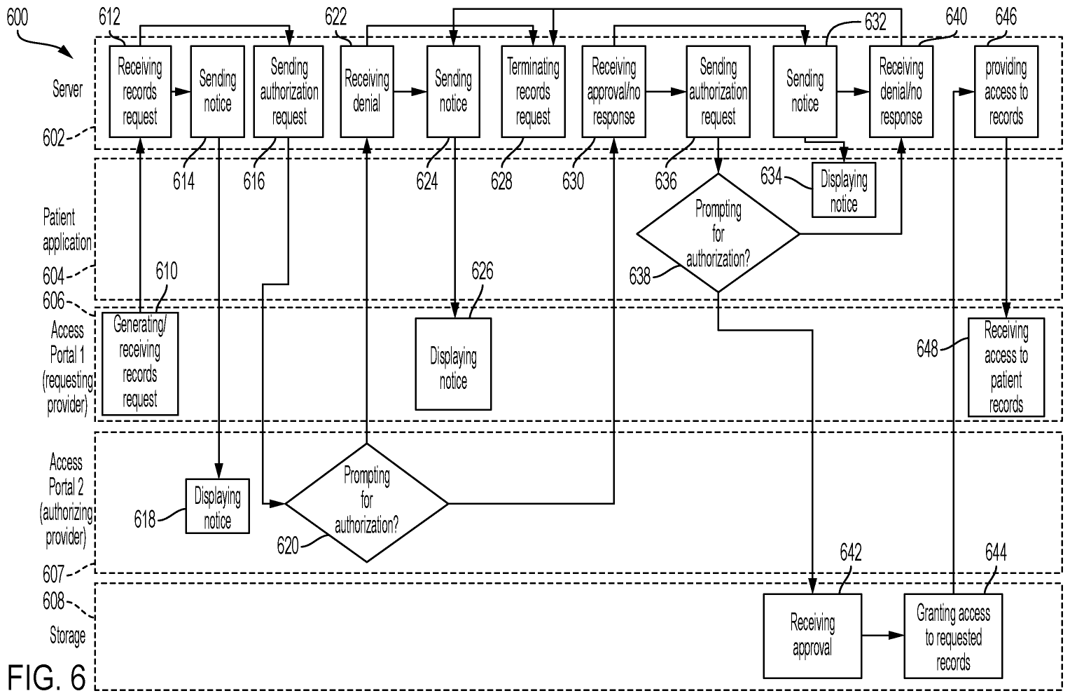

FIG. 6 provides a flowchart depicting an exemplary method of providing a requesting party with access to patient records using a Health ID Network system, namely method 600.

Method 600 comprises: generating/receiving 610 a records request via first access portal 606; receiving 612 the records request from first access portal 606 at server 602; sending 614 a notification concerning the records request to second access portal 607 responsive to receipt of the records request by server 602 at step 612, and displaying 618 the notice sent in step 614 on a provider user device via second patient portal 607; sending 616 an authorization request consistent with the received records request to second access portal 607; and prompting 620 an authorized provider for approval or denial of the authorization request received from server 602 via second access portal 607.

Further, responsive to server 602 receiving 622 a denial of the authorization request from second access portal 607, the system may perform the actions of sending 624 a notification regarding the denial of authorization for the records request to first access portal 606 and displaying 626 said notification on a provider user device via first access portal 606. Alternatively, responsive to server 602 receiving 630 an approval of, or no response to, the authorization request from second access portal 607, the system may proceed by sending 632 a notification of the received records request to patient application 604 and displaying 634 said notification on a patient user device via patient application 604; sending 636 an authorization request consistent with the received records request to patient application 604; and prompting 638 a patient for approval of the authorization request via patient application 604.

In response to server 602 receiving 640 either a denial of the authorization request, or no response to prompting 638, server 602 may perform the actions of sending 624 a notification concerning the lack of approval of the records request to first access portal 606 and displaying 626 said notification on a provider user device via first access portal 606, and of terminating 628 the records request. Otherwise, in response to receiving 642 an approval of the authorization request from patient portal 604 responsive to prompting 638, storage 608 may perform the granting 644 of access to the records stored in storage 608 covered by the records request, and server 602 may take actions required for providing 646 access to records stored in storage 608 to first access portal 606, whereby the requesting party may engage in accessing 648 said records via first access portal 606.

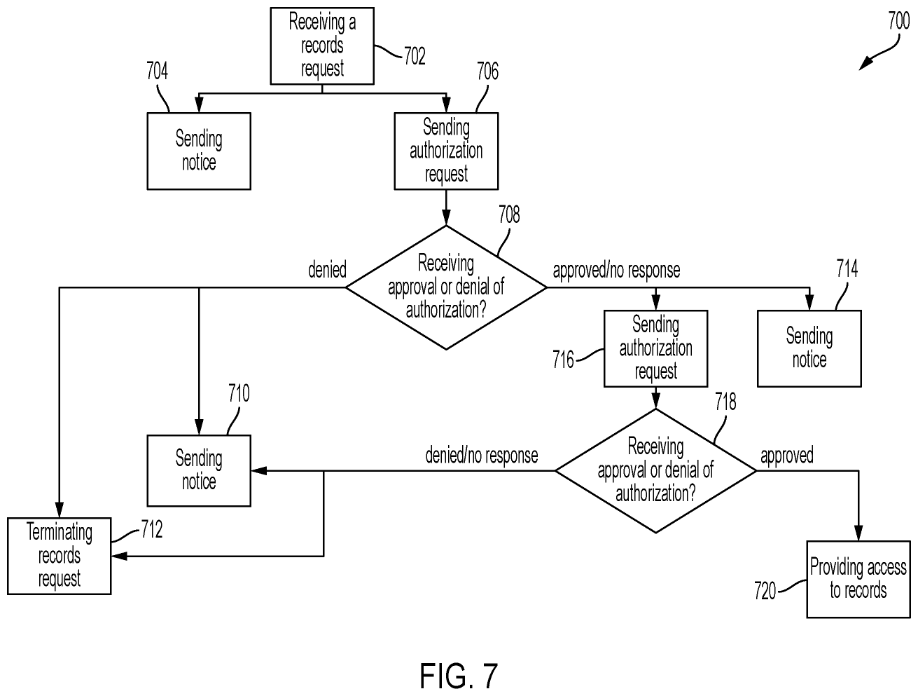

FIG. 7 provides a flowchart depicting an alternate exemplary method of providing a requesting party with access to patient records using a Health ID Network system, as performed from the perspective of a Health ID Network system server, namely method 700.

Method 700 comprises: receiving 702 a records request from a first access portal at a server; sending 704 a notification regrading the records request to a second access portal and sending 706 an authorization request consistent with the received records request to the second access portal responsive to receiving 702; and receiving 708 one of an approval, a denial, or a lack of response from the second access portal responsive to sending 706.

Responsive to receiving 708 comprising a denial of the authorization request, method 700 may further comprise sending 710 a notification regarding the denial of the records request to the first patient portal, and terminating 712 the records request; or responsive to receiving 708 comprising one of an approval of, or no response to, the authorization request, the system may engage in sending 714 a notification regarding the received records request to a patient application, sending 716 an authorization request to the patient application, and receiving 718 one of an approval, a denial, or a lack of response from the patient application responsive to sending 716.

Responsive to receiving 718 comprising one of a denial of, or no response to, the authorization request, the system may proceed by sending 710 a notification regarding the denial of the records request to the first patient portal and terminating 712 the records request. Otherwise, responsive to receiving 716 comprising an approval of the authorization request, the system may perform actions required for providing 720 access to patient health records stored in the system's storage covered by the records request.

In embodiments, the methods to obtain a patient's medical records from a provider, discussed above, may comprise the request for and transfer of medical records that are compliant with the FHIR standard.

In embodiments, methods to obtain a patient's medical records from a provider, may include a mechanism whereby the request for access to records may be automatically denied responsive to one or more predetermined events, including but not limited to, the expiration of a set period of time from the sending of the request for records without receipt of the approvals required for such a transfer to be authorized.

Various embodiments of Health ID Network systems may comprise software which when executed by the system may cause the system to access and monitor a patient's healthcare-related records, such as insurance claims, and automatically collect the patient's electronic health records from a plurality of the patient's providers. Various implementation of such system may achieve the automated collection of a patient's medical records by monitoring the patient's health insurance claims and financial payments to identify medical record holders and then automatically requesting the patient's medical records from the holders so identified, thereby automating the collection and consolidation of those records.

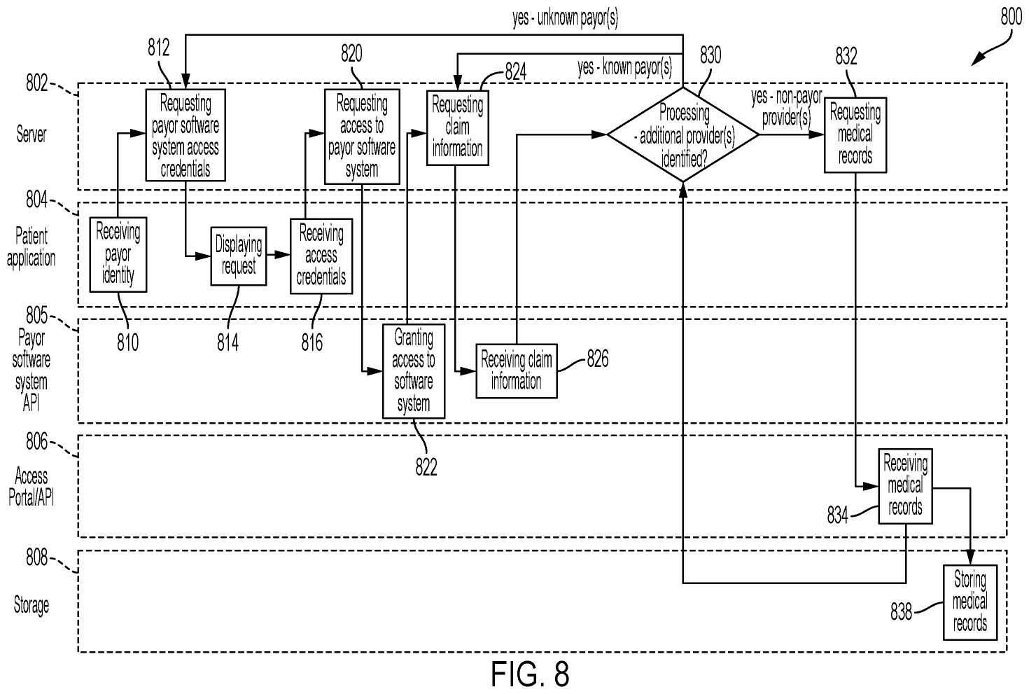

FIG. 8 provides a flowchart depicting an exemplary method for automatically collecting patient health records based on insurance information using a Health ID Network system, namely method 800.

Method 800 comprises receiving 810 the identity of one or more payors via patient application 804. Responsive to receiving a payor identity, server 802 may engage in requesting 812 access credentials for a software system associated with the identified payor. The systems request for access credentials may be displayed 814 to a patient via patient application 804, responsive to which the patient may provide such credentials to the system via patient application 804. After receiving 816 the requested access credentials from the patient via patent application 804, the system may provide those access credentials when requesting 820 access to the payor software system. Upon receiving the granting 822 of access to the payor software system via API 805, the system may proceed with requesting 824 claim information from the payor via API 805 and receiving 826 claim information from the payor via API 805 responsive thereto.

Once the system has received the requested claim information from the payor, it may perform the action of processing 830 the received information to identify additional providers. If additional payor providers are identified by processing 830, steps of method 800 may be iterated using the identity of the payors so identified in the place of the payor identity provided via patient application 804 at step 810. If the identified payors are known to the system, the steps of method 800 may be iterated using such known identified payors starting at requesting step 824; alternatively, if the payors identified at processing step 830 are unknown to the system, method 800 may be iterated using the identity of such unknown payors starting at requesting step 812.

If any additional providers which are not payors are identified at step 830, whether responsive to the initial occurrence of step 830, or to any iteration of step 830, as may be contemplated hereby, method 800 may proceed by requesting 832 medical records from each non-payor provider so identified via access portal/API 806. Upon receiving 834 medical records from one or more provider(s) responsive to said request the system may perform one or more of storing 838 said records in system storage 808 and iterating the steps of method 800 beginning at processing step 830 using the records received at step 834.

In embodiments, the step of receiving 810 payor identity may be facilitated by the server sending a listing of known payors to patient application 804. In embodiments, the listing of such known payors may consist of or comprise payors which are already connected to the Health ID Network system, such as by way of a provider account, an access portal, or an API.

In embodiments, some implementations of such methods for collecting patient medical records via their insurance claims information received from payors may provide for the step of requesting 820 claim information via payor API 805, may be automatically iterated by the system responsive to one or more of various factors, such as but not limited to the passage of a predetermined period of time, or the system's receipt of information indicating a potential change in the status of a claim. Such iterative actions may enable the system to effectively monitor the claims information for changes over time. In such embodiments, the system may be configured, to trigger additional requests for claims information responsive to its determination of a change in claim status, based on such monitoring.

In embodiments, method 800 may further comprise, iterating the steps of requesting 824, receiving 826, and processing 830, responsive to the system identifying new claims information at processing step 830 while monitoring claims received through API 805.

In embodiments, the step of requesting 832 patient medical records from one or more providers identified from the insurance claims information received from the payor may be performed using an electronic request sent from server 802 to a provider via access portal/API 806. In alternate embodiments, that step may be performed manually, such as by way of fax, physical mail, or other suitable means of communication.

In embodiments, the step of processing 830 the received insurance claim information received from the payor portal to identify additional providers, may comprise identifying additional payor providers (e.g., secondary insurance provider) and/or additional claims involving the patient and requesting information from them by reperforming requesting step 824 using such information. Moreover, in such embodiments, if the step of processing 830 information identifies additional payor providers, the steps of method 800 may be iterated, starting at one of requesting steps 812 or 824 for each payor so identified. Such an iterative process may enable the system to automatically expand its collecting of patient records responsive to new information gained from the collection process itself.

In embodiments method 800 may additionally provide for iteration of processing step 830 (and any steps following therefrom) to be performed responsive to receipt of records at step 834, using the records so received.

In embodiments, some implementations of such methods for collecting patient medical records via their insurance claims information received from payors may require the payors to be using the FHIR compliant standards for storage and exchange of records, and/or be connected to the system via a FHIR complaint structure/system, a FHIR aggregator. Accordingly, in embodiments, payors, may submit claims information and/or member data to a FHIR aggregator, which may then be accessed by the Health ID Network system to retrieve the data stored thereon.

It should be understood that, while the foregoing exemplary method of collecting patient information using Health ID Network systems, depicted in and discussed in reference to FIG. 8, has been described the use of insurance claim information received from payors as a means by which the system may identify additional providers relevant to a patient from which the system may then collect additional patient health records, such methods are not limited to the use of such information alone. Embodiments of such methods may use any other suitable patient-related information (e.g., insurance claims history, financial records, mobile device location data, etc.) which may be received from a provider to identify additional providers from which the system may then request and collect patient health records.

Accordingly, more generalized methods of automatically collecting patient health records, such as but not limited to those that utilize information other than health records, such information including but not limited to insurance claims history, financial transaction information, and/or user device location in order to identify new providers and thereby expand the scope of the system's ability to collect patient health records, are contemplated hereby.

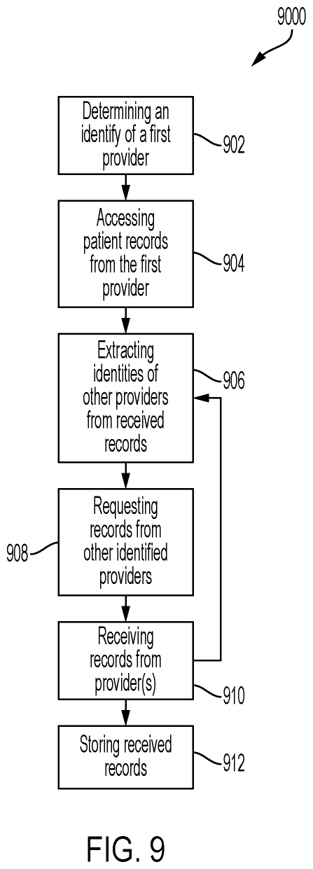

An exemplary embodiment of one such generalized record collection methodology is depicted in FIG. 9, which provides a flowchart depicting an alternate exemplary method for automatically collecting patient health records, as performed from the perspective of a Health ID Network system, namely method 900.

Method 900 comprises a Health ID Network system determining 902 an identity of a first provider. In embodiments, the identity of the first provider may be received from a patient via a patient application and may be responsive to the system prompting the patient with a request for provider information via the patent application. Once the system has determined the identity of a first provider, the system may continue method 900 by accessing 904 patient records from the first provider and extracting 906 identities of other provider(s) from the records so accessed. In embodiments, such patient records may comprise one or more of patient health records, insurance claims records, financial records, and user device location records.

Once the system has identified one or more additional providers via step 906, it may proceed by requesting 908 records from the additional provider(s) so identified. After receiving 910 records from the additional provider(s) responsive to the request made at step 908, the system may store 912 the records in system storage.

In embodiment, method 900 may provide for the steps of extracting 906, requesting 908, and receiving 910 to be iterated using the records received at receiving step 910 in place of those accessed via the first provider at step 904. By permitting iteration of the extraction of additional provider identities from records received by the system and the request for and receipt of records therefrom, which may themselves be processed by the system and which may result in the identification of further additional providers, method 900 may permit the system to recursively expand the scope of its records collection using information gathered from the records that it collects.

In embodiments, the steps of accessing 910 the first provider's records, requesting 908 records from other providers, and receiving 910 records form provider(s) may be performed via one or more access portals and/or APIs operating between the Health ID Network system each of the providers.

Records received by a Health ID Network system may be in one or more of various formats, including those which may be less desirable than others for one reason or another. Accordingly, embodiments of Health ID Network systems may be configured such that, when bringing in information from different formats (e.g., .pdf, .jpg, etc.), the system may automatically process the information from the inbound format, identify relevant patient health information, and convert one or more of the records received and the relevant patient health information identified from those records to a standardized digital format. The standard format to which the records and/or information may be converted may be a format that is more universally acceptable than the original format, and/or which may facilitate the access to and capture of relevant health information therefrom by means known in the relevant art, including but not limited to use of fields and metadata.

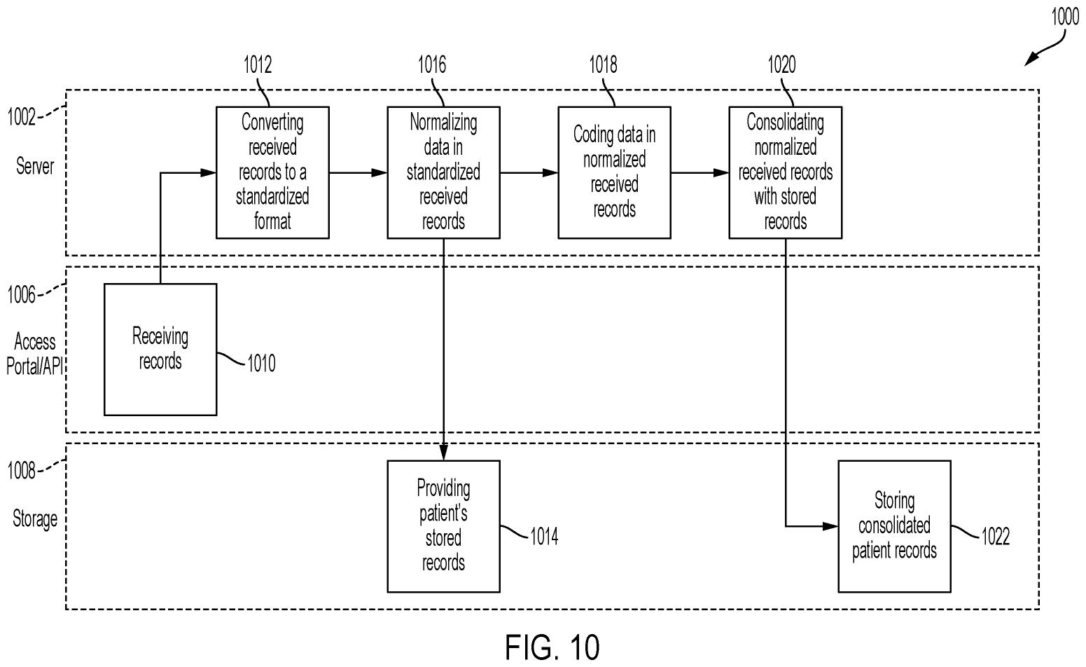

FIG. 10 provides a flowchart depicting an exemplary method for automatically normalizing and consolidating health records received by a Health ID Network system, namely method 1000.

Method 1000 comprises receiving 1010 patient health records via access portal/API 1006 and converting 1012 the received records from the format(s) in which they were received into one or more standardized formats. Once the formats of the received records have been standardized, storage 1008 may provide 1014 server 1002 with access to the patient's records stored in storage 1008 of the system. The received records may then go through normalizing 1016, during which the received records may be compared against the system's stored records for the patient, provided at step 1014, and inconsistencies therebetween may be identified and corrected. Once the received records have been converted to a standardized format and normalized against the systems other records for the patient, the system may then proceed to coding 1016 the standardized and normalized records, whereby the system may tag certain identified information in the records using medical coding systems (such as for diagnosis and/or billing purposes). After such processing has occurred to the received records, the system may then engage in the action of consolidating 1020 the received records with the system's other records for the patient. Consolidating 1020 may include removal of duplicate information from the records and/or the application of business rules for determining what information should be stored in the records. Once the records received at step 1010 have been processed and consolidated with the rest of the system's records associated with the patient, the system may store 1022 the consolidated patient record in system storage 1008.

In embodiments, the standardized formatting into which the system may convert received medical records and/or infotainment extracted therefore, may comprise a formatting compliant with an established standard (e.g., C-CDA, FHIR, HIPPA, etc.).

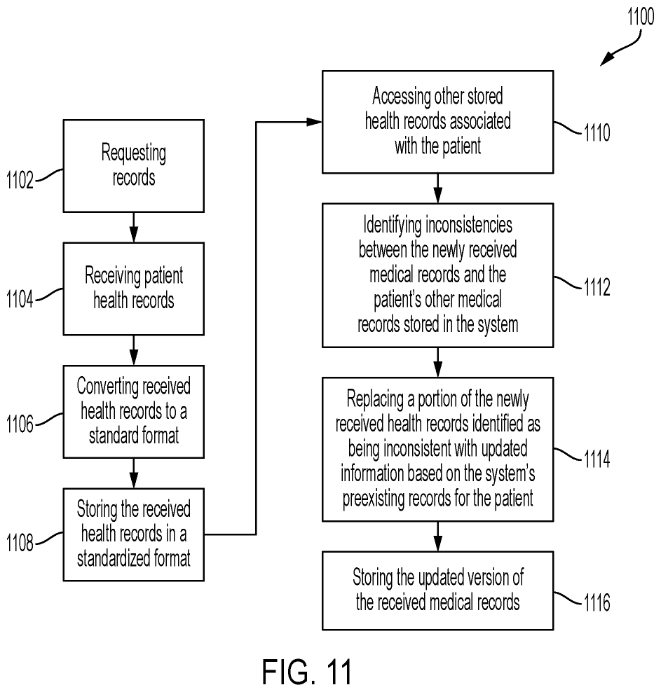

FIG. 11 provides a flowchart depicting an alternate exemplary method for automatically normalizing and consolidating health records received by a Health ID Network system, as may be performed from the perspective of a Health ID Network system server, namely method 1100.

Method 1100 comprises: requesting 1102 patient health records from one or more of a provider via an access portal and/or API, and a patient via a patient application; receiving 1104 patient health records comprising one or more non-standardized formats from one or more of a provider and the patient via one or more of the patient application, an access portal, and an API responsive to requesting 1102; converting 1106 one or more of the records so received from said one or more non-standardized formats to one or more standardized formats; storing 1108 the received records in the standardized format(s) the system's storage; accessing 1110 the patient's other health records and information stored in the system; identifying 1112 inconsistencies between the received patient health records and those which the system had in its possession prior to the step of receiving 1104; replacing 1114 a portion of the received records, identified in step 1112 as being inconsistent with the system's prior records for the patient, with updated information based on such preexisting patient records; and storing 1116 a version of the received records comprising the updated information in the storage.

In embodiments, the step of identifying 1112 inconsistencies between the received patient health records and those which the system had in its possession prior to the step of receiving 1104, may comprise comparing the received records in their standardized format(s) (i.e., post the step of converting 1106 the received medial records to standardized format(s)), against the patient records already stored in the system, as the previously stored records may also be in such standardized format(s), which may simplify step of identifying 1112 inconsistencies therebetween. Alternatively, other embodiments of method 1100, may provide for the step of identifying 1112 inconsistencies between the received patient health records and those which the system had in its possession prior to the step of receiving 1104, to comprise comparing the received records in the format(s) in which they were initially received by the system against the patient records previously stored in the system.

In embodiments, the step of storing 116 may comprise storing a version of the received records comprising the updated information in either a standardized format, or in the original format in which the record was received by the system.

To avoid burdening a patient with the need to sign records transfer authorization forms for each of the patient's previous or current providers, embodiments of the Health ID Network systems may request a master authorization from the patient so that once received, the system may then create authorization having a verified digital identity and transmit such forms along with records requests to each of a plurality of providers, on the patient's behalf.

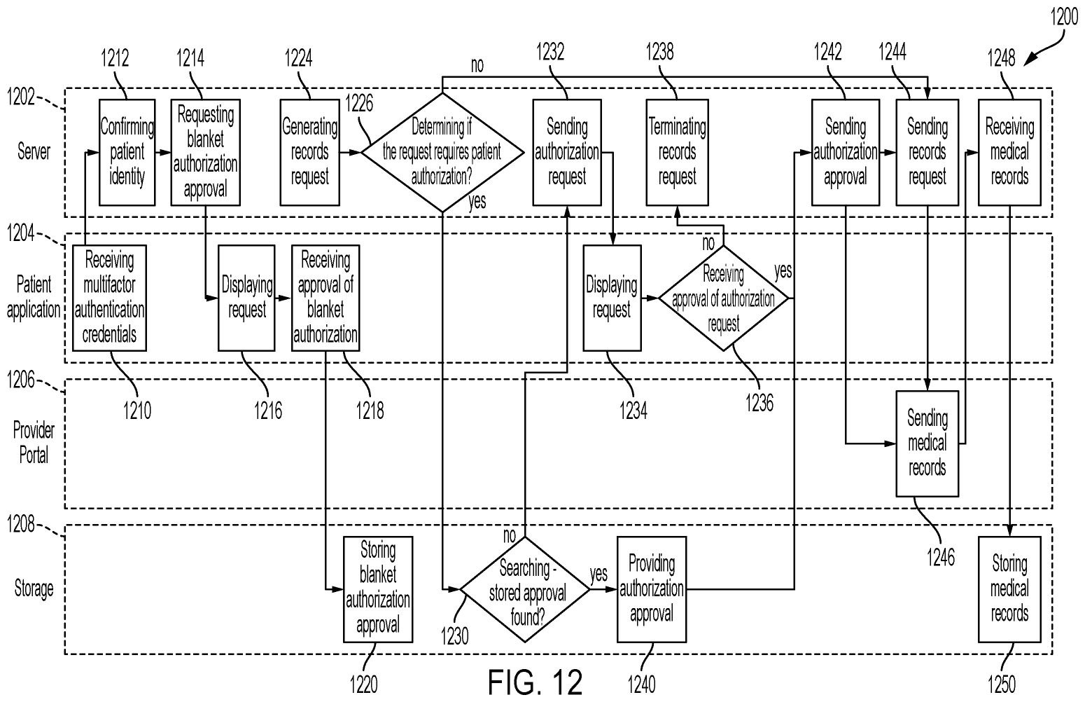

FIG. 12 provides a flowchart depicting a method for implementing an exemplary automated workflow for automatically authorizing requests for the transfer of a patient's medical records to a Health ID Network system, namely method 1200.

Method 1200 comprises: receiving 1210 multifactor authentication credentials from a patient device via patient application 1204; confirming 1212 patient identity using the multifactor authentication credentials received via patient application 1204 at step 1210; requesting 1214 a blanket authorization approval from the patient; displaying 1216 the request for a blanket authorization approval on a patient device via patient application 1204; receiving 1218 patient approval of the blanket authorization via patient application 1204; and storing 1222 the approved blanket authorization in storage 1208.

Method 1200 further comprises server 1202 generating 1224 a request for a patient's medical records and determining 1226 whether or not the records request generated at step 1224 requires patient authorization.

If, at step 1226, the system determines that the records request does not require patient authorization, server 1202 may proceed with sending 1244 the records request to a provider via access portal 1206. If, on the other hand, the system determines at step 1226 that the records request requires patient authorization, the system may proceed by searching 1230 for approvals stored in storage 1208 consistent with that required for the records request.

If the step of searching 1230 for stored approvals is successful and a suitable patient approval is found in storage 1208, storage 1208 may then provide 1240 the stored authorization approval to server 1202, and server may take the action of sending 1242 the stored authorization approval to the provider via access portal 1206 and sending 1244 the records request to the provider via the access portal. If the system is unsuccessful at find a stored patient authorization suitable to meet the requirements of the records request at step 1230, the system may take perform the step of sending 1232 an authorization request for the records request to a patient device via patent application 1204, and patient application 1204 may then operate to display 1234 said authorization request on the patient mobile device. The system may receive 1236 the patient's response to the approval request via patient application 1204. Responsive to patient application 1204 receiving 1236 a denial of the authorization request, or not receiving a response to the request, the system may take the action of terminating 1238 the records request; otherwise, responsive to the patient application 1204 receiving an approval of the authorization request, the system may perform the actions of sending 1242 the authorization approval received from patient application 1204 to the provider via access portal 1206 and sending 1244 the records request to the provider via access portal 1206.

Once the system has performed the step of sending 1244 the records request to the provider via access portal 1206, method 1200 proceed by server 1202 receiving 1248 medical records from a provider via an access portal, API, or other suitable means of communication responsive to the records request sent at step 1244, and once such records are received, may engage in storing 1250 the received medical records in storage 1208.

In embodiments, the multifactor authentication credentials received via patient application 1204 at step 1210 may comprise at least one biometric authentication factor comprising the patient's biometric information so that the party accessing the system via patient application 1204 may be affirmatively and individually identified as the patient, and accordingly, any consents/authorizations/approvals submitted via patient application 1204 may be capable of being verified as coming from the patient.

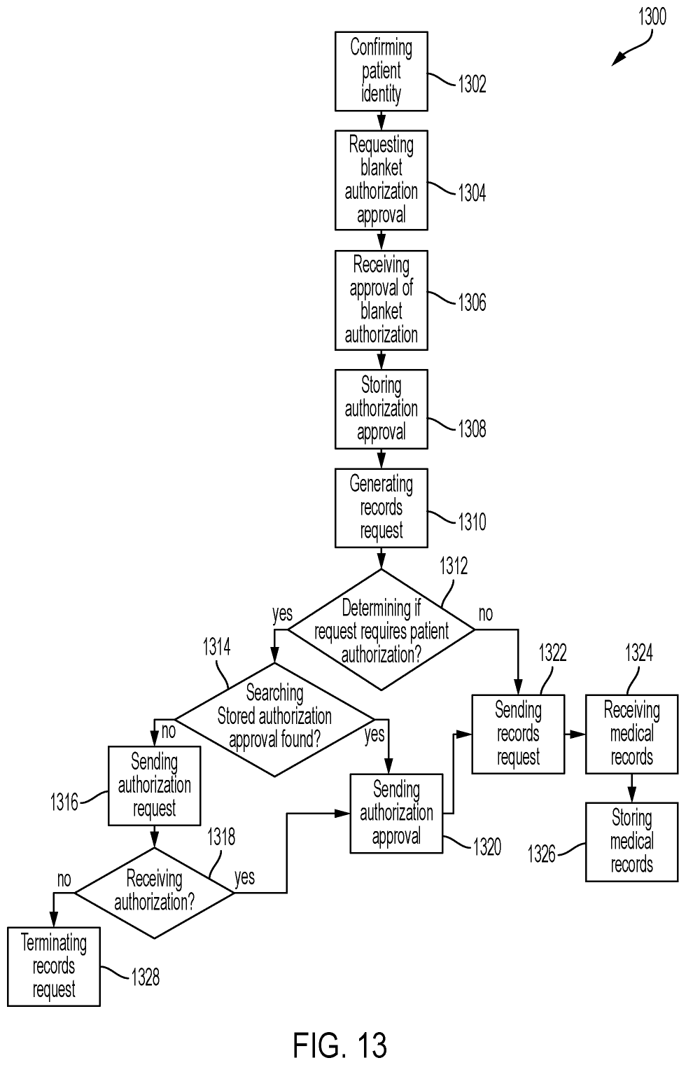

FIG. 13 provides a flowchart depicting an alternate exemplary method for implementing an exemplary automated workflow for automatically authorizing requests for the transfer of a patient's medical records to a Health ID Network system, as performed from the perspective of a Health ID Network system server, namely method 1300.

Method 1300 comprises: confirming 1302 patient identity by comparing multifactor authentication credentials received from a patient device via a patent application against party identity and authentication information stored in the system; requesting 1304 a blanket authorization approval from the party verified as being the patient, via the patient application; receiving 1306 patient approval of the requested blanket authorization via the patient application; storing 1308 the approved blanket authorization in storage associated with the system; generating 1310 a request for medical records on behalf of the patient; and determining 1312 if the records request so generated requires patient authorization.

If the system determines at step 1312 that no patient authorization is required, the system may proceed by sending 1322 the records request to a provider via an access portal, API, or other suitable means of communication. Alternatively, if the system determines at step 1312 that a patient authorization is required for the records request, the system may perform the actions of searching 1314 system storage for stored authorization approvals, such as the blanket authorization approval. If the system finds a stored authorization suitable for the records request the system may proceed by sending 1320 the request authorization found at step 1314 to a provider via one or more of an access portal, an API, or another suitable means of communication, and sending 1322 the records request to the provider along with the request authorization; or if the system fails to find a stored authorization suitable for the records request it may proceed by sending 1316 an authorization request to a patient via the patient application.

If the system ends up receiving 1318 either a denial of, or no response to, its sending 1316 the authorization request via the patient portal, it may proceed by terminating 1328 the records request. If the system ends up receiving 1318 an approval of the authorization request sent to the patient at step 1316 via the patient portal, it may then proceed by sending 1320 the approved authorization request to the provider via one or more of the access portal, an API, or another suitable means of communication, and then may continue to the step of sending 1322 the records request to the provider along with the associated approved authorization request.

Once the system has performed the step of sending 1322 the records request to the provider device, method 1300 may proceed by the system receiving 1324 medical records from a provider via an access portal, API, or other suitable means of communication responsive to the request sent at step 1322, and once such records are received, storing 1326 the received medical records in system storage.

In embodiments, the step of confirming 1302 patient identity may require the credentials submitted via the patient application to comprise a biometric authentication factor requiring the patient's biometric information.

It should be understood that embodiments of Health ID Network systems may be configured so that they may verify the identity of a party accessing the system via one or more of a patient application, access portal, or API, and the extent of that party's authority, responsive to any request received from such a party input to the system. Authentication actions using multifactor authentication protocols and credentials has been discussed herein with respect to such parties initially accessing the system; however, the system may perform such party identity authentication actions at any time, including responsive to any request or other input received by the system from the applicable user device, including but not limited to, when a party makes a records request, approves an authorization request, or otherwise responds to a query/prompt from the system. Similarly, the system may be configured to perform the steps required to verify an identified party's authorization to perform any given action, responsive to a request by the party to perform such an action. Further, it should be understood that any of the methods discussed hereinabove or otherwise contemplated by this disclosure may comprise such authentication and authorization checks at suitable times, including responsive to the receipt of input from a user device via any of the patient application, an access portal, or an API. By providing for such authentication and/or authorization checks to be performed at any time, and especially responsive to inputs being received by the system from user devices, Health ID Network systems may ensure confirmation that the party submitting a request, providing authorization, etc. possesses the valid authority to do so.

Additionally, embodiments of the Health ID Network systems such as those described in detail hereinabove may be operable to help facilitate highly trustworthy real-time health screening by enabling a patient to access their authenticated medical records from a user device via the patient application.

While the present systems and methods have been disclosed according to preferred embodiments of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise. The term “connected” means “communicatively connected” unless otherwise defined.

When a single embodiment is described herein, it will be readily apparent that more than one embodiment may be used in place of a single embodiment. Similarly, where more than one embodiment is described herein, it will be readily apparent that a single embodiment may be substituted for that one device.

In light of the wide variety of authentication and transaction management systems known in the art, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

To aid the Patent Office and any readers of any patent issued on this application in interpreting the claims appended hereto, the applicant wishes to note that it does not intend any of the appended claims or claim elements to invoke 35 U.S.C. 112(f) unless the words “means for” or “step for” are explicitly used in the particular claim.

We claim:

1. A healthcare information exchange system comprising:
a server, a storage device, a network and a user device, wherein the server is configured with a software application for facilitating communication between the server, the storage device and the user device via the network, and wherein the storage device is configured to receive, record, and store information associated with a patient account;
wherein the server is configured with:
an authentication module configured to authenticate an identity of a party requesting access to the information associated with a patient account;
an authorization module configured to determine if the party requesting authorization is authorized to access the information associated with a patient account by comparing authorization information received from the user device through which the request is made against stored authorization information;
a request module configured to create records requests, to receive records requests via the software application, and to transmit records requests to a user device via the network;
a consolidation module configured to receive medical information associated with the patient, convert the received medical information from an original format to a standardized, consolidated format;
a normalization module configured to compare the received medical information against the stored information associated with the patient account, to identify inconsistencies between the received medical information and the stored information associated with a patient account, and to correct the inconsistencies in the received medical information so identified based on the stored information associated with a patient account; and
a recordation module configured to record health information to the information associated with a patient account on the storage device; and
wherein the healthcare information exchange system is configured such that requests for information received by the server are passed to the authentication module after being processed by the request module, are passed to the authorization module responsive to a positive authentication at the authentication module; and
wherein the healthcare information exchange system is configured such that health information received by the server is passed to the normalization module after being processed by the consolidation module, are passed to the recordation module after being processed by the normalization module and are recorded to the information associated with a patient account on the storage device after being processed by the recordation module.

2. The healthcare information exchange system of claim 1, wherein the authentication module is configured to authenticate an identity of a party requesting access to the information associated with a patient account in response to receipt of a request to access the information associated with a patient account by comparing an authentication credential received from a user device making the request against stored access information, and to deny access to the information associated with a patient account if the authentication module cannot authenticate the identity of a party requesting access.

3. The healthcare information exchange system of claim 1, wherein the authorization module is configured to determine if a party requesting authorization is authorized to access the information associated with a patient account by comparing authorization information received from the user device through which the request is made against stored authorization information, denying access to a restricted portion of the information associated with a patient account comprising information for which the received authorization information failed to meet stored authorization information associated with the information being requested; and providing access to a permitted portion of the information associated with a patient account comprising information for which the received authorization information met or exceeded the authorization information associated with the information being requested.

4. The healthcare information exchange system of claim 1, wherein the software application comprises one or more of a patient application, an API, and an access portal, and wherein the software application is hosted by the server.

5. The healthcare information exchange system of claim 1, wherein the software application comprises a patient application operating on the user device.

6. The healthcare information exchange system of claim 3, wherein the authentication information received from the user device making the request for information and the access information each comprises a multifactor authentication credential.

7. The healthcare information exchange system of claim n-1, wherein each of the multifactor authentication credential comprises a biometric authentication factor.

8. The healthcare information exchange system of claim 1, wherein responsive to the authorization module determining that the that a party submitting the request does not have authorization to access the information included in the request.

9. The healthcare information exchange system of claim 8, wherein the authorization module is further configured to generate and transmit an authorization to a patient or an authorizing provider via the software application; and performing one of granting access to the information included in the request responsive to receipt of an approval of the authorization request from the patient or authorizing provider, or continuing to deny access to the information.
